(19) United States
(12) Patent Application Publication
(10) Pub. No.: US 2010/0297765 A1
(43) Pub. Date: Nov. 25, 2010

(54) METHOD FOR PRODUCING LANGERHANS CELLS OR INTERSTITIAL DENDRITIC CELLS OR BOTH FROM CD14+ MONOCYTES

(75) Inventors: Nicolas Bechetoille, Saint Genis-Laval (FR); Valerie Andre, Ampuis (FR); Isabelle Orly, Irigny (FR); Eric Perrier, Les Cotes D'arey (FR)

Correspondence Address: BASF Beauty Care Solutions France;C/O Connolly Bove Lodge & Hutz LLP, 1007 North Orange Street, P.O. Box 2207, Wilmington, DE, 19899, US

(73) Assignee: BASF Beauty Care Solutions France S.A.S., Lyon (FR)

(21) Appl. No.: 12/851,592

(22) Filed: Aug. 6, 2010

Related U.S. Application Data
(63) Continuation of application No. 11/897,617, filed on Sep. 1, 2007, now abandoned.

(30) Foreign Application Priority Data
Sep. 4, 2006 (FR) 0653657

(51) Int. Cl. C12N 5/0784; C12N 5/0786

(52) U.S. Cl. 435/377; 435/325; 435/395

(57) ABSTRACT

The present invention relates to a method for preparing Langerhans cells or interstitial dendritic cells or both from CD14⁺ monocytes stemming from the peripheral circulatory blood of a living being, wherein the method comprises differentiation of CD 14⁺ monocytes into either Langerhans cells, interstitial dendritic cells, or into both types of cells by placing the CD14⁺ monocytes in the presence of a cell environment comprising epithelial cells and/or mesenchymatous cells.

The present invention also relates to cell or tissue models comprising such prepared Langerhans cells and/or interstitial dendritic cells, and optionally macrophages and endothelial cells, and to the uses of such cell or tissue models.

### CROSS-REFERENCE TO PRIORITY APPLICATION

This application is a continuation of U.S. patent application Ser. No. 11/897,617, filed Sep. 1, 2007, which claims benefit under 35 U.S.C. §119 of French patent application No. 0653657, filed Sep. 4, 2006. Both of these applications hereby are expressly incorporated herein by reference in their entirety.

### BACKGROUND OF THE INVENTION

This invention relates to a method for producing dendritic cells, such as Langerhans cells and/or interstitial dendritic cells and/or dermal dendritic cells, from CD14⁺ monocytes isolated from peripheral circulatory blood. This invention also relates to tissue models prepared with such cells, and the use of such tissue models for testing active ingredients and/or studying biological/biochemical phenomena involved in skin tissue.

Dendritic cells (also referred to herein as “DCs”) are cells having antigens described as sentries of the immune system. Indeed, they have a quasi-ubiquitous localization, i.e., in the thymus, systemic circulation, secondary lymphoid organs and also peripheral tissues such as the skin and the mono-stratified or pluri-stratified mucosae. Although there are a very small number of them in the organism, the DCs are at the centre of the triggering of specific immune responses, by exerting a control on the specificity, the intensity and the nature of the immune response and are placed at the interface between innate and acquired immunity. In addition to their function for igniting the immune response, the DCs also play a role in inducing peripheral tolerance.

The precursors of DCs stem from the differentiation of CD34⁺ haematopoietic progenitors just as many populations of the immune system and blood cells. They are conveyed via the blood at the skin and the mucosae in order to differentiate and reside there as immature DCs. This immature state is expressed by a characteristic phenotype and a strong functional capability of capturing antigens. Two types of peripheral DCs are described according to their in vivo localization:
The Langerhans cells (also referred to herein as “LCs”) are localized at the epithelia of the malpighian type (skin and mucosae) where they specifically express langerine which is a lectin of types C. Langerine is involved in the formation of an intracytoplasmic organite, Birbeck's granule which is the specific ultrastructural and reference label of the LCs. LCs also express characteristic labels such as CD 1a and HLA-DR.
Interstitial DCs (referred to herein as “IDCs”) are located at the lamina of mucosae, for example at the dermis of the skin. Interstitial DCs which are located at the dermis also are called dermal DCs (referred to herein as “DDCs”). Thus, in the description which follows this type of cell is generally referred to interstitial dendritic cells, or IDCs, independently of whether they are at the lamina of the mucosae or at the dermis of the skin.
These cells share many similarities and common labels with the cell line of monocytes/macrophages. In the dermis of the skin, DCCs specifically express DC-SIGN but also characteristic labels such as HLA-DR, FXIIIa, MMR and CD 1a. Following the capture of an antigen, the LCs and IDCs migrate towards the lymph nodes in order to present the antigenic information to the lymphocytes T. This migration which is correlated with activation of LCs and IDCs is expressed by phenotypic and functional changes. For example, the “activated” LCs and IDCs acquire the expression of CD80 and CD86 which are co-stimulation labels and acquire the expression of the CCR7 receptor which is absolutely necessary to skin migration of the cells. At the lymph nodes, the activated LCs and IDCs acquire a phenotype of “mature” or “interdigitated” DCs from their interaction with T lymphocytes. This mature condition is synonym with a characteristic phenotype such as for example the expression of CD83 and DC-LAMP and with a strong allostimulating capacity i.e. the capability of inducing proliferation of T lymphocytes. Because of their capability of migrating towards proximal nodes after having captured an exo-antigen, the LCs and IDCs are responsible for many skin pathologies such as contact allergies and have recently been described as the first targets of the human immunodeficiency virus (HIV).

An interesting use of LCs and IDCs stemming from skin or human mucosae, especially when combined with epithelial cells or mesenchymatous cells of the fibroblast type, consists of integrating them into three-dimensional organotypical cultures such as:
Models of “reconstructed epidermises” and of “reconstructed mucosae”, for example of the vaginal and oral type, exclusively integrating LCs (Régnier et al., JID 1997; 109:510-2; European Patent No. 0 789 074; Sivard et al., Exp. Dermatol. 2003; 12:346-55),
Models of “reconstructed dermis” and of “reconstructed chorion”, integrating DDCs or IDCs (Guironnet et al., JID 2001; 116:933-9 and Dumont et al., AIDS Res. Hum. Retroviruses 2004; 20:383-97).

Today, these uses are extremely limited because: (1) the absence of a fast, simplified and inexpensive industrial scale method for producing in vitro LCs and IDCs and, (2) of the imperfection of the described prior art 3D models which exclusively show the epithelial portion or the conjunctive portion of the reconstructed tissue and but not both of the associated tissue compartments.

The articles of Régnier et al., Sivard et al. and Dumont et al. deal with the use of CD34⁺ precursors stemming from umbilical cord blood which has the following major drawbacks: (1) the number of isolated CD34⁺ progenitors is limited since they are extracted from umbilical cord blood which is a limited source, and are therefore difficult to use industrially; (2) the prior culture (requiring 6 to 12 days) of the CD34⁺ progenitors before their integration into the 3D models which is done in order to induce their differentiation into LCs or IDCs; and (3) the requirement of these described methods that exogenous cytokines be added to the media utilized for growing reconstructed epidermises, reconstructed mucosae and reconstructed chorions integrating the LCs or some IDCs.

In the article of Guironnet et al., the authors have generated DCCs from blood monocytes in order to integrate them into a reconstructed dermis model. Similar to those limitations discussed above, two drawbacks of the Guironnet method need to be highlighted: (1) the prior culture of monocytes (for 6 days) before their integration into the reconstructed dermis that is required in order to induce their differentiation into DCCs; and (2) the addition of exogenous cytokines in the media for growing the reconstructed dermis integrating the DCCs. In this same article, the authors also show that monocytes directly integrated into the equivalent dermis and without adding any cytokines, do not differentiate into DCs.

U.S. Pat. No. 6,130,482 describes the co-culture of keratinocytes and precursors of LC in an adequate nutritious medium in order to carry out differentiation of the precursors into LCs, notably with the purpose of integrating them into an epidermis model in order to assess the synthesizing irritating or allergenic potency of a product. However, this patent describes the use of CD34⁺ haematopoietic progenitors stemming from umbilical cord blood with reference to the method described by Caux et al., Nature, 1992 Nov. 19, 360(6401):258-61, which comprises the growing of CD34⁺ haematopoietic progenitors in the presence of exogenous cytokines. U.S. Pat. No. 6,130,482 generally defines the precursors of LCs as being able to express CD1a⁺, but only the use of CD34⁺ haematopoietic cells as CD34⁺ precursors of LCs is described therein. The CD34⁺ haematopoietic cells are not very numerous in the peripheral blood and do not allow development of an industrially satisfactory differentiation method. Further, the use of precursors stemming from umbilical cord blood is unsatisfactory as this blood is not available in a large amount.

More recently, French patent 2 833 271 B1 describes the differentiation of CD14⁺ monocytes in order to obtain LCs, IDCs, some LCs and some DCIs simultaneously, which may then be grown in the presence of epithelial cells and/or mesenchymatous cells stemming from skin or human mucosae. The following tissue models are described in this patent:
Models of “reconstructed epidermis” and of “reconstructed mucosae” exclusively integrating LCs,
Models of “reconstructed dermis” and of “reconstructed chorion” integrating DCCs or IDCs,
Models of “reconstructed skins” and of “reconstructed mucosae” integrating LCs and IDCs simultaneously.

As is the issue with the other prior art methods described above, a drawback of the method described in French patent 2883271 is the need for prior growing of monocytes (i.e., for 6 days) in the presence of exogenous cytokines before integrating them into 3D models, which is done in order to induce their differentiation into LCs, into IDCs, or into LCs and IDCs simultaneously.

Of the above described methods, only the method of Patent FR 2 833 271 B1 describes the differentiation of monocytes into LCs and/or into IDCs with the purpose of providing LCs and/or IDCs for developing immunocompetent tissue models close to normal human skin. Unexpectedly, it has now been found that this method may be further improved by using a simple, not very costly, and fast differentiation method.

Thus, none of the existing art provides all of the advantages and benefits of the present invention.

### OBJECT OF THE INVENTION

With the invention, it is possible for the first time to solve each of the technical problems set forth earlier, in a safe, reliable, and reproducible way and in a manner which may be used on an industrial and commercial scale and preferably on an industrial scale in the agro-feeding, medical, pharmaceutical, or cosmetic industries.

The present invention allows for generating LCs, or IDCs or LCs and IDCs simultaneously, or IDCs and macrophages and endothelial cells simultaneously or LCs and IDCs and macrophages and endothelial cells simultaneously, from a unique living precursor which stems from peripheral circulatory blood.

The present invention further provides epidermis, epithelium, dermis, chorion, skin or mucosa models containing the aforementioned cells (LC, IDC/DCC, etc.), said models being of the best possible quality in order to reproduce an epidermis, an epithelium, a dermis, a chorion, a skin or a mucosa of a living being, preferably of a mammal, and more preferably of a human being. A further object of the present invention is to provide epithelial and/or conjunctive sheets as well as immunocompetent equivalents of skin or of mucosae.

The present invention further provides a method for differentiating monocytes stemming from peripheral circulatory blood in order to obtain the aforementioned cells (LC, IDC/DCC, etc.)

The present invention also provides skin models which may be utilized as an alternative method to animal experimentation, particularly for testing the irritating and/or sensitizing potency of a cosmetic ingredient.

Another object of the present invention is to provide tissue models as described above for testing cosmetic, pharmaceutical or dermo-pharmaceutical active ingredients, and particularly for assessing any or all of their activity, toxicity or pharmacotoxicity.

Yet another object of the present invention is to provide tissue models as described above for testing molecules or chemicals, and is especially useful for assessing their toxicity.

An additional object of the present invention is to provide tissue models as described above which are useful for investigating biological/biochemical phenomena at intercellular and intracellular level.

A still further object of the invention is to provide a tissue model which may be utilized as a research tool for pharmacotoxicological investigation, for example to conduct tests in vitro for predicting the allergizing/irritating/sensitizing power of external agents.

Another object of the present invention is to provide a tissue model which may be utilized as a research tool for investigating substances having immunomodulating properties.

The invention also relates to the use of the models as described above in tissue or cell engineering, particularly for repairing at least one portion of the tissues of a living being.

These and other features, aspects, and advantages of the present invention will become evident to those skilled in the art from a reading of the present disclosure.

### SUMMARY OF THE INVENTION

All percentages and ratios used herein are by weight of the total composition, unless otherwise designated. All publications cited herein are incorporated by reference in their entirety.

Within the scope of the invention, when discussing “cells”, this always refers to “living cells”, unless stated otherwise.

As used herein, “peripheral circulatory blood”, means the blood of any living being having a blood system in which the blood accomplishes a circuit, notably at the periphery, preferably in mammals, and more preferably in a human being.

As used herein, “active ingredient”, includes any substance, product or composition capable or potentially capable of providing a positive benefit to a product in either the agro-feeding, food, dermo-pharmaceutical, pharmaceutical, or cosmetics industry. For example, a substance, product or composition capable or potentially capable of inhibiting an enzyme known to reduce collagen elasticity would be potentially capable of providing a positive benefit to an anti-aging cosmetic product and would thus be considered an active ingredient for the cosmetics industry.

As used herein, “addition of exogenous cytokine” means the addition of at least one cytokine in addition to the cytokines synthesized by the cells present in the relevant cell medium.

The invention therefore relates to differentiation of CD14⁺ monocytes, in the presence of epithelial cells and/or mesenchymatous cells, including mesenchymatous cells of the fibroblast type, into:
LCs,
IDCs,
LCs and IDCs simultaneously,
IDCs and macrophages and/or endothelial cells simultaneously,
LCs and IDCs and macrophages and/or endothelial cells simultaneously.

The present invention relates to differentiation of CD14⁺ monocytes into LCs and/or IDCs (without prior growing of the monocytes under conditions promoting their differentiation, and without prior growing with exogenous cytokines) in the presence of epithelial cells and/or mesenchymatous cells, for example of the fibroblast type, in order to obtain a cell model comprising LCs and/or IDCs, their culture being preferably performed essentially without significant addition of exogenous cytokines, meaning without adding any cytokine in addition to the endogenous cytokines synthesized by the cells present in the medium.

The invention notably relates to a method for differentiating CD14⁺ monocytes into LCs and/or IDCs, comprising the following steps:
collecting CD14⁺ monocytes from circulatory blood, preferably the circulatory blood of a human being or other mammal;
maintaining the collected CD14⁺ monocytes under conditions which do not promote their differentiation into DCs;
placing the collected CD14⁺ monocytes into contact in a cell environment comprising epithelial cells and/or mesenchymatous under conditions which do not promote differentiation of the CD14⁺ monocytes into LCs, or IDCs, or LCs and IDCs. Preferably the epithelial cells are keratinocytes and the mesenchymatous cells are fibroblasts.

The above step for maintaining CD14⁺ monocytes under conditions which do not promote their differentiation into DCs is preferably limited in time to the time needed in order to carry out sowing of the CD14⁺ monocytes shortly or even directly after they have been collected from the circulatory blood.

Advantageously, conditions which do not promote differentiation of CD14⁺ monocytes into DCs may be obtained through a culture which does not comprise any exogenous cytokine. However, one skilled in the art will recognize that the purpose of the culture conditions is that the CD14⁺ monocytes do not engage in DC differentiation paths, and thus may utilize other culture conditions known in the art which may limit or prevent such differentiation.

Through the use of the present invention, it is possible to obtain a gain in the number of precursors/cells as the present invention avoids the need to grow the CD14⁺ monocytes prior to differentiation, a step which is accompanied by a certain level of cell mortality.

DCs generated in vitro are very sensitive and fragile cells. The first parameter to be checked is the yield in the obtained cells. It is obvious, but not quantifiable, that the loss and/or cell mortality of DCs is significant when the latter are sown in any cell culture model. The use of CD14⁺ monocytes according to the present invention, as precursors of DCs, remedies this problem and thus allows a better yield in cells.

With the present invention, it is also possible to obtain a gain in time as the differentiation of monocytes into LCs or IDCs generally requires six days of culture.

With the present invention it is further possible to obtain a gain in reagents as preferably no exogenous cytokine is used.

The quality of the generated cells may also be increased through the use of the present invention. In vitro generation of DCs from monocytes is related to a cell differentiation phase under the effect of immune soluble mediators which are cytokines. It has been described that DCs of the LC and DDC type derived from monocytes are not “synchronous” in their cell differentiation process (Bechetoille, et al., Journal of Leukocytes Biology). In other words, the CD14⁺ monocytes which have initiated their differentiation generate LCs and DCCs which do not all have the same differentiation stage. This imperfection may be corrected by avoiding this differentiation step as is accomplished by the present invention. The monocytes grown in the present invention are influenced by the cell, cytokine and matrix environment of the cell reconstructions, which forms a more physiological environment for differentiation of DCs.

It is well known that DCs generated in vitro have the capability of “maturating” spontaneously, which is a major problem as mature DCs may no longer be activated and stimulated. Although the immature conditions of these cells in vitro may be controlled, the risk of spontaneous maturity in vitro may be dispensed with by directly using monocytes as precursors of LCs and DCCs. Also, the advantageous use of cell or tissue culture models according to the present invention as a system for differentiation of CD14⁺ monocytes into DCs is more physiological as it better reproduces the natural conditions for differentiation of DCs in the skin and in the mucosae and immature DCs which are comparable to their homologs in vivo may thereby be obtained.

In this same context, by using CD14⁺ monocytes as precursors of DCs, as opposed to the use of DCs generated beforehand, it is possible to generate in the present models for growing phenotypically more immature and functionally more sensitive LCs and DCCs. Indeed, and in this context, it has been described that DCs derived from CD34⁺ progenitors and then grown in a reconstructed chorion in the presence of exogenous cytokines has a more satisfactory immaturity condition, in terms of replication of the HIV virus, than its homologs grown in the presence of exogenous cytokines (Dumont, et al., AIDS Res. and Hum. Retroviruses 20: 383-397, 2004). The obtained DCs are very different from DCs obtained by the prior art methods described previously, including FR 2 833 271 B1.

Further, direct use of CD14⁺ monocytes as precursors of skin DCs is a more physiological process as compared with the process described earlier. Indeed, in vivo in the organism, blood monocytes colonize the skin, where the cell, cytokine and matrix environment controls their differentiation. Integration of DCs differentiated beforehand in the present culture models was an interesting alternative which presently is less satisfactory than directly using their precursors which are the CD14⁺ monocytes. This improvement of the system is immunohistologically expressed by a more homogenous cell distribution of LCs and DCCs in the three-dimensional culture models of the present invention.

Advantageously, with the present invention, it is possible to freeze freshly isolated monocytes before their use in the models.

With the present invention, it is also possible to generate cells having substantially the same phenotype and the same functions as their in vivo homologs.

The present invention notably relates to a unique cell precursor which, when it is co-cultivated:

In an epithelial environment, differentiates phenotypically and/or functionally into LCs,

In a conjunctive environment differentiates phenotypically and/or functionally into IDCs, macrophages and endothelial cells.

### DESCRIPTION OF THE INVENTION

Thus, the present invention according to a first aspect, relates to a method for preparing LCs, or IDCs, or LCs and IDCs, from CD14⁺ monocytes stemming from peripheral circulatory blood of a living being, preferably of a human being, comprising differentiation of CD14⁺ monocytes into LCs or into IDCs or into LCs and IDCs by putting CD14⁺ monocytes in presence with a cell environment comprising epithelial cells, such as keratinocytes, and/or mesenchymatous cells, such as dermal fibroblasts.

Advantageously, differentiation of CD14⁺ monocytes enables one to obtain IDCs and macrophages and endothelial cells, or LCs, IDCs, macrophages and endothelial cells. Advantageously, the distribution of the cell population of LCs and IDCs is a function of the cell type which is jointly grown with monocytes. The use of keratinocytes promotes differentiation into LCs and the use of fibroblasts promotes differentiation into IDCs.

Advantageously, the differentiation of the monocytes is carried out without significant addition of any exogenous cytokine. According to one particularly preferred embodiment, no exogenous cytokine is added.

Advantageously, growing monocytes with an epithelial cell environment, such as keratinocytes, promotes differentiation of the monocytes into immature and functional LCs. As used herein, “immature” means that the LCs do no, or only very slightly, express activation labels (CD80, CD86, CCR7) and maturation labels (CD83, DC-LAMP). On the other hand, immature LCs express CCR6 and “functional” means the LCs are provided with antigene internalization capacities (immature condition), cell migration capacities (immature and activated condition), and antigenic presentation capacities (mature condition).

According to one preferred embodiment, the culture of monocytes with a mesenchymatous cell environment, such as an environment with dermal fibroblasts, promotes differentiation of the monocytes into immature and functional IDCs. Reference is made to the definitions of “immature” and “functional” above, with the exception that immature IDCs do not express CCR6.

Advantageously, the culture of monocytes with a cell environment comprising epithelial cells, such keratinocytes, and mesenchymatous cells, such as dermal fibroblasts, promotes differentiation of the monocytes into typical LCs and IDCs. As used herein, “typical” means close to their homologs in vivo in terms of immature phenotype and functionality, i.e., of their capabilities of reacting after stimulation, stress, etc.

The proportion of epithelial cells and/or mesenchymatous cells, relative to the CD14+ monocytes, used for the culture (for the differentiation) depends on the cell distribution between LCs and/or IDCs and epithelial cells and/or mesenchymatous cells, which one skilled in the art wishes to obtain and one skilled in the art would select the proportion accordingly.

The invention according to a second aspect relates to a method for growing CD14⁺ monocytes, the growing method comprising the integration into a cell or tissue model of CD14⁺ monocytes stemming from peripheral circulatory blood of a living being, preferably of a mammal, and more preferably of a human being, said cell or tissue model comprising epithelial cells, preferably keratinocytes, or mesenchymatous cells, preferably dermal fibroblasts, or both epithelial cells and mesenchymatous cells, in order to obtain differentiation of CD14⁺ monocytes within the model into LCs, or IDCs, or into LCs and IDCs, by growing CD14⁺ monocytes in the presence of such epithelial cells or mesenchymatous cells or both.

Advantageously, the growing of monocytes is carried out without adding any exogenous cytokine.

Advantageously, the cell or tissue model is selected from the group consisting in an epidermis model, an epithelium model, a dermis model, a chorion model, a skin model or a model of a mucosa, in particular of a gingival or vaginal mucosa.

Advantageously, the three-dimensional culture model comprises a dermal or chorion matrix support selected from the group consisting of:

a collagen- or fibrin-based gel or film comprising mesenchymatous cells, in particular fibroblasts,

a porous matrix which is made from collagen which may contain one or more glycosaminoglycans and/or optionally chitosan, these porous matrices either integrating mesenchymatous cells, in particular fibroblasts, or not,

an inert support selected from the group consisting of a semi-permeable synthetic membrane, in particular a semi-permeable nitrocellulose membrane, a semi-permeable nylon membrane, a polytetrafluoroethylene (TEFLON®, PTFE), a membrane or sponge, a semi-permeable polycarbonate or polyethylene terephthalate (PET) membrane, an inorganic membrane with a capillary porous structure of aluminum oxide (semi-permeable Anopore membrane), a semi-permeable polyester membrane, said inert support either containing mesenchymatous cells, in particular fibroblasts, or not.

an inert support treated for polycarbonate- or polystyrene-based culture, said inert support either containing mesenchymatous cells, in particular fibroblasts, or not.

Advantageously, the tissue model used comprises said dermal or chorion support on which epithelial cells, preferably keratinocytes, have been deposited at the surface.

Advantageously, the cell or tissue model comprises at least one additional cell type, for example nerve cells and/or endothelial cells and/or melanocytes and/or lymphocytes and/or adipose cells and/or cutaneous annexes, such as bristles, hairs, sebaceous glands.

Advantageously, a portion of the CD14⁺ monocytes differentiates into endothelial cells and macrophages, in particular when they are put into a cell or tissue model comprising at least mesenchymatous cells.

Advantageously, the method mainly comprises LCs, or IDCs, or a mixture of LCs and IDCs, or a mixture of LCs, IDCs, endothelial cells and macrophages, or a mixture of IDCs, endothelial cells and macrophages.

Advantageously, the cell or tissue model comprises an epithelial portion on a conjunctive matrix and is characterized in that the majority of the population of LCs are localized in the epithelial portion, and in that the majority of the IDCs, the macrophages and the endothelial cells are localized in the conjunctive matrix.

The invention according to a third aspect, relates to a cell model comprising at least one of said LC and/or IDC populations, and further optionally comprising a population of macrophages and/or endothelial cells, capable of being obtained according to a method as defined earlier.

Said model is characterized in that the obtained DCs are different from those obtained according to the methods described earlier, the main differences being concerned with synchronization of the cell differentiation steps for LCs and IDCs.

The invention according to the fourth aspect, relates to a tissue model, comprising at least one of said LC and/or IDC populations, and further optionally comprising a population of macrophages and/or endothelial cells, capable of being obtained according to a method as defined earlier, said tissue model being selected from the group consisting of an epidermis model, an epithelium model, a dermis model, a chorion model, a skin model, or a model of a mucosa, in particular of a gingival or vaginal mucosa.

Advantageously, the cell or tissue model described above is immunocompetent.

Advantageously, the tissue model comprises an epithelial portion comprising epithelial cells, such as keratinocytes, and a conjunctive matrix comprising mesenchymatous cells, such as dermal or chorion fibroblasts, said model being characterized in that the LCs are essentially localized in the epithelial portion, and in that the present IDCs and macrophages and/or endothelial cells are essentially localized in the conjunctive matrix.

Said model is characterized in that the obtained DCs are different from those obtained according methods described earlier, the main differences being concerned with the more homogenous cell distribution of LCs and DCCs in the present three-dimensional culture models.

The invention according to a fifth aspect relates to frozen CD14⁺ monocytes, isolated from the peripheral circulatory blood of a living being, in particular of a human being.

In particular with the invention, it is possible to generate populations of different DCs, the different functionalities of which may account for the whole of the phenomena involved in the organism's defense/infection processes, such as irritation, allergenicity and sensitization phenomena.

Thus, the invention according to sixth aspect, relates to the use of at least one cell or tissue model as defined earlier as an investigation model in the field of cosmetics, dermo-pharmacy or pharmacy, and/or for active ingredient selection.

Another interesting use of LCs and/or IDCs is the utilization for assessing the irritating versus the sensitizing potency of new molecules. European directive 2003/15/EC, which prohibits since 2005 the use of animals for assessing the toxicity of a cosmetic finished product, strongly urges public and industrial laboratories to develop in vitro or in silico predictive methods for predicting the sensitizing potency of new molecules. In this context, because of their key role, in triggering contact allergy, the use of cutaneous DCs as an alternative method to animal experimentation, is today an axis of development.

The invention according to a seventh aspect relates to the use of at least one cell or tissue model as defined earlier, for investigating phenomena occurring in the organism's defense/infection processes, and activity, in particular the immuno-stimulating or immunosuppressive activity, of an active ingredient or for assessing or for inducing immunomodulation (immunotolerance or immunoactivation) by said active ingredient, or for conducting in vitro tests for predicting the allergizing/irritating/sensitizing potency of external agents or for investigating toxicity of molecules or chemicals.

The invention according to an eighth aspect relates to the use of at least one cell or tissue model as defined earlier, for investigating the physiopathology of epithelial barriers; irritation of the skin and mucosae; aggressions of a biological nature such as for example viruses, retroviruses, such as HIV, bacteria, fungi, micro-organisms, particle antigens; phototoxicity; photoprotection; the effect of active ingredients, in particular of cosmetic or pharmaceutical ingredients; the effect of finished products, in particular of cosmetic or pharmaceutical products; the effect of molecules or chemicals; the mechanisms for infection by a pathogenic agent.

Advantageously, this invention relates to investigating toxicity of active ingredients or other substances. Preferably this investigation is carried out by studying cell labels, for example those labels of DCs.

The invention according to a ninth aspect relates to the use of at least one cell or tissue model as defined earlier, for investigating mechanisms involved in the phenomena of viral infection, replication and transmission of viruses, including retroviruses like HIV, or for investigating and developing alternative therapeutic methods, including the administration of vaccines or drugs.

The invention according to a tenth aspect relates to the use of at least one cell or tissue model as defined earlier for detecting the presence of a pathogenic agent such as for example viruses, retroviruses, such as HIV, bacteria, fungi, micro-organisms, particle antigens.

The invention according to an eleventh aspect relates to the use of at least one cell or tissue model as defined earlier, for a medical, biomedical, or cosmetic application, in particular for modulating the immune or tolerance response in vitro or in vivo, as a result of an environmental aggression, in particular of the physical type, such as UV irradiation, of the chemical type, such as irritating/allergizing/sensitizing agents, of the biological type, in particular with a preventive or curative therapeutic purpose.

The invention according to a twelfth aspect relates to the use of at least one cell or tissue model as defined earlier, for cell or tissue engineering applications, for medical or biomedical applications, for example in anti-cancer cell therapy, for example by a DCs injection capable of stimulating the immune response; for example in cell therapy in the case of an auto-immune disease, for example by creating an immunotolerance stimulation, for example by producing anergic T cells; for example in gene therapy of diseases affecting the immunitary system; or for developing and making vaccines.

The invention according to a thirteenth aspect relates to a method for making a tissue model comprising:
isolation of CD14⁺ monocytes from peripheral circulatory blood of a living being,
sowing skin or mucosal cells on a support and growing them in a nutritious medium in order to obtain reconstructed tissue,
sowing in the reconstructed tissue CD14⁺ monocytes either simultaneously or not with skin or mucosal cells,
growing the reconstructed tissue comprising CD14⁺ monocytes and skin or mucosal cells under conditions allowing differentiation of CD14⁺ monocytes into LCs, into a mixture of IDCs, endothelial cells and macrophages, into a mixture of LCs, IDCs, endothelial cells and macrophages, the skin cells being epidermal keratinocytes when the reconstructed tissue is an epidermis model, the skin cells being dermal fibroblasts when the reconstructed tissue is a dermis model, the mucosal cells being epithelial mucosal cells when the reconstructed tissue is an epithelium model, the mucosal cells being mucosal fibroblasts when the reconstructed tissue is a chorion model.

The epithelial cells are for example isolated from at least one skin tissue.

Sowing of the skin or mucosal cells may be carried out before sowing or after having sown CD14⁺ monocytes.

Preferably, sowing of CD14⁺ monocytes is carried out simultaneously with the sowing of skin or mucosal cells.

The invention according to a fourteenth aspect relates to a method for making a reconstructed skin or a reconstructed mucosa comprising an epithelial portion comprising keratinocytes or epithelial mucosal cells, optionally in the presence of Merkel cells and/or melanocytes, and a conjunctive matrix comprising dermal or mucosal fibroblasts, said method comprising:
sowing and growing in a nutritious medium, keratinocytes or epithelial mucosal cells, possibly in the presence of Merkel cells and/or melanocytes, at the surface of a dermis or chorion model, comprising IDCs and optionally endothelial cells and macrophages, said dermis or chorion model being able to be obtained by a method for making a dermis or chorion model as defined above,
sowing and growing either simultaneously with keratinocytes or epithelial mucosal cells or not, CD14⁺ monocytes isolated from peripheral circulatory blood of a living being in the presence of keratinocytes or epithelial mucosal cells under conditions allowing differentiation of CD14⁺ monocytes into LCs.

Advantageously, the cells are cells of a human being. Preferably, these methods for making tissue models do not comprise any addition of exogenous cytokine.

Other objects, features and advantages of the invention will become clearly apparent to one skilled in the art after reading the explanatory description which refers to examples which are only given as an illustration and which may by no means limit the scope of the invention.

The examples are an integral part of the presence invention and any feature which appears to be novel relatively to any prior state of the art, from the description taken as a whole, including the examples, is an integral part of the invention in its function and in its generality.

Thus, each example has a general scope.

On the other hand, in the examples all the percentages are given by weight, unless stated otherwise, and the temperature is expressed in degrees Celsius, unless stated otherwise, and the pressure is atmospheric pressure, unless stated otherwise.

### EXAMPLES

### Example 1

### Methods for Separating Monocytes from Peripheral Circulatory Blood

The peripheral circulatory blood was harvested by sampling venous blood on one or more human donors, preferably in vacutainers or bags supplemented with usual anti-coagulant products such as lithium heparin.

Separation of monocytes from circulatory blood may advantageously be performed according to the following protocols:

1. After centrifuging blood on a lymphocyte separation medium, the mononucleated cells are recovered, and then:
Either labeled with a cocktail of antibodies, such as for example anti-CD3, anti-CD7, anti-CD16, anti-CD19, anti-CD56, anti-CD123 antibodies, anti-glycophorin A coupled with magnetic beads. After passing over a magnetized column, only the non-labeled monocytes are eluted and recovered,
Or labeled with a specific antibody of the monocytes, such as an anti-CD14 antibody, coupled with magnetic beads; after passing over a magnetic column, only the labeled monocytes are retained in the column. After elution from the column, the labeled monocytes are recovered.
Or labeled with a specific antibody of the monocytes, such as an anti-CD16 antibody, coupled with a fluorochrome, such as phycoerythrin. After cell sorting in flux cytometry, only the labeled monocytes are recovered.

2. The monocytes are recovered by proceeding with any physical separation method well-known to one skilled in the art and notably by sedimentation or centrifugation and they are eluted as such for subsequent cultures.

3. For 100 mL of sample blood, up to about 150 million (±20 million) mononucleated cells are extracted and up to 40 million monocytes are purified.

### Example 2

### Method for Freezing Monocytes Isolated from Peripheral Circulatory Blood

The monocytes, obtained as described in Example 1, are suspended in a nutritious medium, for example RPMI medium, supplemented with serum and a cryoprotective agent, such as DMSO (dimethyl sulfoxide), and then frozen.

When thawing out monocytes, cell mortality is less than 30%.

For 100 mL of sample peripheral circulatory blood, up to 80.10.sup.6 monocytes are frozen, and up to 76.10⁶ monocytes are recovered after thawing them out.

### Example 3

### A Pluricellular Monolayer Model of Keratinocytes and LCs in a Co-Culture

Monocytes were obtained according to the process of Example 1 or 2.

1 to 2.10⁶ human keratinocytes and 1 to 2.10⁶ human monocytes (obtained according to Example 1 or 2) are jointly grown in a nutritious medium, for example of the K-SFM type, in culture dishes, for example of the 6-well plate type. The joint culture is maintained for 6 days in a nutritious medium, for example of the K-SFM type, without adding any exogenous cytokine.

The cells are then recovered by an enzymatic method well-known to one skilled in the art and notably by trypsination. 2.10⁵ cells of the mixed cell suspension consisting of keratinocytes and monocytes are incubated with a monoclonal anti-Langerine antibody, and then analyzed in flux cytometry. We observe up to 40% of Langerine⁺ LCs.

### Example 4

### Pluricellular Monolayer Model of Fibroblasts and DDC in a Co-Culture

Monocytes were obtained according to the process of Example 1 or 2.

1 to 2.10⁶ human fibroblasts and 1 to 2.10⁶ human monocytes (obtained according to Example 1 or 2) are jointly grown in a nutritious medium, for example of the FBM type, in culture dishes for example of the 6-well plate type. The joint culture is maintained for 6 days in a nutritious medium, for example of the FBM type, without adding any exogenous cytokine.

The cells are then recovered by an enzymatic method well-known to one skilled in the art and notably by trypsination. 2.10⁵ cells of the mixed cell suspension consisting of fibroblasts and monocytes are incubated with a monoclonal anti-DC-SIGN antibody, and then analyzed in flux cytometry. We observe up to 60% of DC-SIGN+ DCCs.

### Example 5

### The Use of Monolayer Pluricellular Models Described in Examples 3 and 4 for Investigating the Profile of Cytokines Secreted Under the Effect of an Active Ingredient

In order to assess the irritating, sensitizing, allergizing potency, and to estimate a possible pro- or anti-inflammatory activity of an active ingredient intended for human skin, we quantify in the culture supernatants, secretion of cytokines, for example, IL-1, IL-6, IL-8, IL-10, TNF-□ INF□, according to the following protocol:
The model is made according to the protocol described in Example 3 or 4.
Retinol is then added into the culture medium at a final concentration of 0.05% for 3 days.
The culture supernatants are then recovered and analyzed.

It is observed that retinol causes stimulation of secretion of pro-inflammatory cytokines.

### Example 6

### The Use of Pluricellular Monolayer Models Described in Examples 3 to 4 for Investigating the Immunoactivating or Immunosuppressive Activity of an Active Ingredient

In order to assess the capacity of the LCs and DCCs of inducing immune and/or tolerogenic responses towards an active ingredient or not, we studied their phenotype profile by flux cytometry, according to the following protocol:
The model is made according to the protocol described in Example 3 or 4.
The active ingredient is then added into the culture medium at various concentrations and for 3 days.
The cells are then recovered by trypsination. 2.10⁵ cells of the mixed cell suspension consisting of keratinocytes and LCs or of fibroblasts and DCCs are incubated in an antibody battery: anti-CCR7, anti-HLA-DR, anti-CD80, anti-CD83, anti-CD86, anti-DC-LAMP.

With cell phenotyping, the immunoactivating (induction and/or increase of the expression of labeled molecules) or immunosuppressive profile (inhibition and/or suppression of the expression of labeled molecules) of the tested active ingredients may be established.

### Example 7

### The Use of Pluricellular Monolayer Models Described in Examples 3 to 4 for Investigating the Immunomodulating Activity of an Active Ingredient

The immunomodulating effect of an active ingredient after an irritating, sensitizing or allergizing stress is investigated according to the following protocol:
The model is made according to the protocol described in Example 3 or 4.
300 μl of TNP (2,4,6-trinitrobenzene-sulfonic acid) are then added at a concentration of 5 mM for 30 mM at 37° C.
After this stimulation, the active ingredients tested at various concentrations are added into the culture medium for 2 days.
The culture media comprising the cells are then recovered in order to investigate the secretion of immune cytokines, for example IL-12; the cells are recovered and then incubated either with an anti-Langerine antibody or with an anti-DC-SIGN antibody in order to sort the LCs or the DCCs in flux cytometry, respectively.
The sorted LCs or DCCs are then sown in migration chambers of the Boyden type (porosity of the membrane from 5 to 8 .mu.m either covered with MATRIGEL™ or not) and then incubated for up to 72 hrs at 37° C.
The number of LCs or DCCs having migrated is quantified, for example by counting in optical microscopy.

The results of migration and/or IL-12 synthesis and secretion tests enable the immunomodulating profile of the active ingredients tested to be established.

### Example 8

### Three-Dimensional Pluricellular Reconstructed Epidermis Model Containing LCs

Monocytes were obtained according to the process of Example 1 or 2.

The model was made according to the following protocol:
0.5 to 1.10⁶ keratinocytes of normal human skin were sown in inserts of the Boyden chamber type (membrane with porosity 0.μm) and then grown in a nutritious medium of the DMEM-Glutamax type supplemented with calf serum, ascorbic acid and preferably a final concentration of 1 mM of EGF (epidermal growth factor) and preferably at a final concentration of 10 ng/mL, hydrocortisone and preferably at a final concentration of 0.4 μg/mL, umulin and preferably at a final concentration of 0.12 IU/mL, isuprel and preferably at a final concentration of 0.4 μg/mL, triiodothyronine and preferably at a final concentration of 2.10⁻⁹ M, adenine and preferably at a final concentration of 24.3 μg/mL, normocin and preferably at a final concentration of 100 μg/mL, for 2 days.
Next 1 to 5.10⁴ monocytes were sown at the surface of the epithelium equivalent, which was grown for a further period of 2 days.
The cultures were then placed at the air-liquid interface for a further period of 10 days in the same medium used for the immersion culture, except for calf serum, hydrocortisone, umulin, isuprel and triiodothyronine.
The cultures were then embedded in an amorphous resin, such as TISSUE-TECK®, and directly frozen in liquid nitrogen.
Immunohistochemical investigations were then conducted on histological sections in order to characterize the cell types by means of specific antibodies.
The performed labelings showed the presence of LCs (Langerine⁺ cells).

Thus, the monocytes integrated into the reconstructed epidermis model differentiated into DCs of the LC type as demonstrated by observation of the lectin, Langerine.

### Example 9

### Three-Dimensional Pluricellular Pigmented and/or Nervous Reconstructed Epidermis Model Containing LCs

Monocytes were obtained according to the process of Example 1 or 2.

The model was made according to the protocol described in Example 8, by simultaneously sowing together 0.5 à 1.10⁵ melanocytes and/or Merkel cells stemming from normal human skin with keratinocytes.

In addition to the labelings described in Example 8, labeling of the melanocytes (HMB45) and a DOPA reaction was carried out in order to detect melanin as well as labeling with an anti-keratin 20 antibody for identifying Merkel cells. Thus, the monocytes integrated into the pigmented and/or nervous reconstructed epidermis model differentiated into DCs of the LC type as demonstrated by observation of the lectin, Langerine.

### Example 10

### Three-Dimensional Pluricellular Epithelium Model of Gingival and Vaginal Reconstructed Mucosae Containing LCs

Monocytes were obtained according to the process of Example 1 or 2.

The model was made according to the protocol described in Example 8 with the following changes:
Normal human keratinocytes were replaced with epithelial cells of normal human gingival and vaginal mucosae.
The percentage of serum in the culture medium was 1%.
Growing was totally performed with immersion in the medium.
The achieved labelings show the presence of LCs (Langerine⁺ cells).

Thus, the monocytes integrated into the epithelium model of gingival and vaginal reconstructed mucosae differentiated into DCs or the LC type as demonstrated by observation of the lectin Langerine.

### Example 11

### Three-Dimensional Pluricellular Reconstructed Skin Model

This model is the association of a reconstructed dermis culture on which an additional culture of a reconstructed epidermis is performed.

The reconstructed dermis model was made according to the following protocol:
0.5 to 1.10⁶ fibroblasts of normal human skin was sown on a matrix substrate based on cross-linked collagen with diphenyl-phosphoryl azide, and then grown in a nutritious medium, DMEM-Glutamax supplemented with 10% calf serum, ascorbic acid and preferably at a final concentration of 1 mM, EGF (epidermal growth factor) and preferably at a final concentration of 10 ng/mL, normocin and preferably at a final concentration of 100 μg/mL, for 14 days.

The reconstructed skin model was made according to the following protocol:
0.5 to 1.10⁶ normal human keratinocytes were sown on the dermal equivalent, and then grown in a nutritious medium, DMEM-Glutamax/Ham F-12 (ratio 3/1 v/v) supplemented with calf serum, ascorbic acid and preferably at a final concentration of 1 mM, EGF (epidermal growth factor) and preferably at a final concentration of 10 ng/mL, hydrocortisone and preferably at a final concentration of 0.4 .μg/mL, umulin and preferably at a final concentration of 0.12 IU/mL, isuprel and preferably at a final concentration of 0.4 pg/mL, triiodothyronine and preferably at a final concentration of 2.10⁻⁹ M, adenine and preferably at a final concentration of 24.3 μg/mL, normocin and preferably at a final concentration of 100 μg/mL. The growing continued for 7 days in the immersed condition. The cultures were then placed at the air-liquid interface for 14 additional days in the same medium as the immersion culture, except for calf serum, hydrocortisone, isuprel, triiodothyronine and umulin. Thus, in vitro reconstruction of both cell compartments of the skin, the epidermis and the dermis, was obtained.

### Example 12

### Three-Dimensional Pluricellular Reconstructed Dermis Model Containing DCCs Macrophages and Endothelial Cells

Monocytes were obtained according to the process of Example 1 or 2.

Growing the reconstructed dermis was performed according to Example 11.

The model is made according to the following protocol:
1 to 5.10⁴ monocytes were sown at the surface of the dermal equivalent, which was grown for a further period of 7 days,
The cultures were then embedded in an amorphous resin, such as Tissue-Teck™, and then directly frozen in liquid nitrogen.
Immunohistochemical investigations were carried out on histological sections in order to characterize the cell types present by means of specific antibodies.
The performed labelings showed the simultaneous presence of DDC (DC-SIGN+ cells), macrophages (CD68⁺ cells) and endothelial cells (CD31⁺ and CD36⁺ cells).

Thus, the monocytes integrated and then grown in our reconstructed dermis model are capable of simultaneously differentiating into DCs of the DDC type, into macrophages and endothelial cells.

### Example 13

### Three-Dimensional Pluricellular Reconstructed Chorion Model Containing IDCs, Macrophages and Endothelial Cells

Monocytes were obtained according to the process of Example 1 or 2.

The model was made according to the protocol described in Example 12 with the following changes:
The fibroblasts were fibroblasts stemming from normal human gingival and vaginal mucosae.
The performed labelings showed the simultaneous presence of IDCs (DC-SIGN+ cells), macrophages (CD68⁺ cells) and endothelial cells (CD31⁺ and CD36⁺ cells).

Thus, the monocytes integrated and then grown in our reconstructed chorion model of the gingival and vaginal type are capable of simultaneously differentiating into DCs of the IDC type, into macrophages and endothelial cells.

### Example 14

### Three-Dimensional Pluricellular Reconstructed Skin Model Containing LCs

Monocytes were obtained according to the process of Example 1 or 2.

The culture of the reconstructed skin model was performed according to Example 11.

The model was made according to the following protocol:
1 to 5.10⁴ monocytes were jointly sown with keratinocytes on the dermal equivalent.

The performed labelings show the presence of LCs (Langerine+ cells and Birbeck+ granules) in the epidermal compartment of the cultures.

Thus, the monocytes grown with keratinocytes in our reconstructed skin model differentiate into DCs of the LC type.

### Example 15

### Three-Dimensional Pluricellular Pigmented and/or Nervous Reconstructed Skin Model Containing LCs

Monocytes were obtained according to the process of Example 1 or 2.

Growth of the reconstructed skin model containing LCs was performed according to Example 14.

The model was made according to the following protocol:
1 to 5.10⁴ melanocytes and/or Merkel cells stemming from normal human skin were jointly sown with keratinocytes and monocytes on the reconstructed dermis.
In addition to the labelings described in Example 14, labeling of the melanocytes (HMB45) and a DOPA reaction was carried out in order to detect melanin, as well as labeling with an anti-keratin 20 antibody for identifying Merkel cells.

The labeling demonstrated that the monocytes grown with keratinocytes, melanocytes and/or Merkel cells in our reconstructed skin model differentiate into DCs of the LC type.

### Example 16

### Three-Dimensional Pluricellular Reconstructed Mucosa Model Containing LCs

Monocytes were obtained according to the process of Example 1 or 2.

The model was made according to the protocol described in Example 14 with the following changes:
Keratinocytes were replaced with epithelial cells of normal human gingival and vaginal mucosae, and the fibroblasts are fibroblasts stemming from normal human gingival and vaginal mucosae.
The percentage of calf serum in the culture medium for the epithelialization step was 1%.
Growth was totally accomplished in immersion in the medium

The performed labelings showed the presence of LCs (Langerine+ cells and Birbeck+ granules) in the epithelial compartment of the cultures. Thus, the monocytes grown with epithelial cells of the gingival or vaginal type in our reconstructed mucosa model differentiate into DCs of the LC type.

### Example 17

### Three-Dimensional Pluricellular Reconstructed Skin Model Containing DCCs, Macrophages and Endothelial Cells

Monocytes were obtained according to the process of Example 1 or 2.

Growth of the reconstructed skin model was achieved according to Example 11.

The model was made according to the following protocol:
1 to 5.10⁴ monocytes were sown at the surface of the dermal equivalent which was grown for a further period of 7 days.

The performed labelings showed the simultaneous presence of DCCs (DC-SIGN+ cells), macrophages (CD68⁺ cells) and endothelial cells (CD31⁺ and CD36⁺ cells). Thus, the monocytes integrated into the dermal compartment of the reconstructed skin model and grown therein differentiate into DCs of the DDC type, into macrophages and endothelial cells, simultaneously.

### Example 18

### Three-Dimensional Pluricellular Pigmented and/or Nervous Reconstructed Skin Model Containing DCCs, Macrophages and Endothelial Cells

The model was made according to the protocol described in Example 17, by simultaneously sowing together 1 to 5.10⁴ melanocytes and/or Merkel cells stemming from normal human skin with keratinocytes.

In addition to the labelings described in Example 17, labeling of melanocytes (HMB45) and a DOPA reaction was carried out in order to detect melanin, as well as a labeling with anti-keratin 20 antibody for observing Merkel cells. The labeling demonstrated that the monocytes integrated into the dermal compartment of the pigmented and/or nervous reconstructed skin model and grown therein differentiate into DCs of the DDC type, into macrophages and endothelial cells simultaneously.

### Example 19

### Three-Dimensional Pluricellular Model of Reconstructed Mucosae Containing IDCs Macrophages and Endothelial Cells

Monocytes were obtained according to the process of Example 1 or 2.

The model was made according to the protocol described in Example 17 with the following changes:
The keratinocytes were replaced with epithelial cells of normal human gingival and vaginal mucosae, and the fibroblasts are fibroblasts stemming from normal human gingival and vaginal mucosae.
The percentage of calf serum in the culture medium for the epithelialization step was 1%.
Growth was totally performed in immersion in the medium.

The performed labelings showed the simultaneous presence of IDCs (DC-SIGN+ cells), macrophages (CD68⁺ cells) and endothelial cells (CD31⁺ cells and CD36⁺). The monocytes integrated into the conjunctive compartment of the reconstructed mucosa model of the gingival and vaginal type and grown therein differentiate into DCs of the IDC type, into macrophages and into endothelial cells, simultaneously.

### Example 20

### Three-Dimensional Pluricellular Reconstructed Skin Model Containing LCs, DCCs, Macrophages and Endothelial Cells

Monocytes were obtained according to the process of Example 1 or 2.

The model was made according to the following protocol:
The reconstructed dermis model containing DCCs, macrophages and endothelial cells was made according to Example 12.
The reconstructed skin model containing LCs was made according to Example 14.

The performed labelings showed the simultaneous presence of LCs (Langerine+ cells and Birbeck+ granules) in the epidermal compartment of the cultures, and of DCCs (DC-SIGN+ cells), macrophages (CD68⁺ cells) and endothelial cells (CD31⁺ and CD36⁺ cells) in the dermal compartment of the cultures. Thus, the monocytes successively grown in the dermal compartment and then with keratinocytes in the reconstructed skin model differentiate into DCs of the LC type in the epidermal compartment and into DCs of the DDC type, into macrophages and endothelial cells, in the dermal compartment, simultaneously.

### Example 21

### Three-Dimensional Pluricellular Pigmented and/or Nervous Reconstructed Skin Model Containing LCs, DCCs, Macrophages and Endothelial Cells

Monocytes were obtained according to the process of Example 1 or 2.

The model is made according to the following protocol:
The reconstructed dermis model containing DCCs, macrophages and endothelial cells is made according to Example 12.
The pigmented and/or nervous reconstructed skin model containing LC1s is made according to Example 15.

The performed labelings detect simultaneous presence of LCs (Langerine+ cells and Birbeck granules) in the epidermal compartment of the cultures, and of DCCs (DC-SIGN+ cells), macrophages (CD68+ cells) and endothelial cells (CD31+ and CD36+ cells) in the dermal compartment of the cultures. Thus, the monocytes successively grown in the dermal compartment and then simultaneously with keratinocytes, melanocytes and/or Merkel cells in the pigmented and/or nervous reconstructed skin model differentiate into DCs of the LC type in the epidermal compartment and into DCs of the DDC type, into macrophages and into endothelial cells in the dermal compartment simultaneously.

### Example 22

### Three-Dimensional Pluricellular Reconstructed Mucosa Model Containing LCs, IDCs, Macrophages and Endothelial Cells

Monocytes were obtained according to the process of Example 1 or 2.

The model was made according to the following protocol:
The reconstructed chorion model containing IDCs, macrophages and endothelial cells was made according to Example 13.
The reconstructed mucosa model containing LCs was made according to Example 16.

The performed labelings showed the simultaneous presence of LCs (Langerine+ cells and Birbeck+ granules) in the epithelial compartment, and the presence of IDCs (DC-SIGN+ cells), macrophages (CD68⁺ cells) and endothelial cells (CD31⁺ and CD36⁺ cells) in the conjunctive compartment of the cultures. Thus, the monocytes successively cultivated in the conjunctive compartment and then with epithelial cells in the reconstructed mucosa model of the gingival of vaginal type differentiate into DCs of the LC type in the epithelial compartment and into DCs of the IDC type, into macrophages and endothelial cells in the conjunctive compartment simultaneously.

### Example 23

### Use of the Three-Dimensional Pluricellular Reconstructed Skin Model Described in Example 20 for Investigating the Influence of Solar UV Radiation

In order to investigate the influence of various environmental factors and in particular of solar UV radiation, the migration and the phenotype profile of LCs and DCCs was assessed in the reconstructed skin model by immuno-histochemical investigations, according to the following protocol:
Models were made according to protocol described in Example 20
On the 42^{nd} day of culture, the reconstructed skins were irradiated at a single dose of 526 kJ/cm² of solar energy corresponding to 2 J/cm² of UVA and 0.5 J/cm² of UVB (solar irradiator Suntest CPS+, ATLAS). Growth continued for a further period of 2 days.
The immuno-histochemical investigations were then made with an antibody battery (anti-CD1a, anti-CD80, anti-CD83, anti-CD86, anti-CCR7, anti-DC-LAMP, anti-DC-SIGN, anti-Langerine, anti-HLA-DR) in order to observe migration of the LCs and DCCs and to characterize their phenotype condition.

After solar UV irradiation, migration of the LCs was observed in the dermal compartment of the cultures, as well as acquisition of the expression of the CCR7 receptor on the C1s and DCCs. Further, only the LCs which have migrated into the dermal compartment, expressed the maturation label DC-LAMP. The pluricellular model of the present invention is therefore predictive of UV stress by the targeting of LCs and DCCs which have migration, activation and maturation capability.

### Example 24

### The use of the Three-Dimensional Pluricellular Reconstructed Skin Model Described in Example 20 for Investigating the Efficiency of Active Ingredients

In order to assess the anti-inflammatory potency of active ingredients intended for human skin, the secretion of pro-inflammatory cytokines, for example IL-1, IL-6, IL-8, TNF-alpha and INF-gamma, was quantified in the culture supernatants according to the following protocol:
Models were made according to the protocol described in Example 20.
On the 42^{nd} day of culture, the reconstructed skins were irradiated with a unique dose of 526 kJ/cm² of solar energy corresponding to 2 J/cm² of UVA and 0.5 J/cm² of UVB (solar irradiator Suntest CPS+, ATLAS).
Subsequently, 8 μl of a cosmetic formulation which contained either 3% anti-oxidant active for example FLAVAGRUM™ (Hesperitine Laurate, ENGELHARD) and FLAVENGER™ (Quercitine Caprylate, ENGELHARD) or not, were applied on the reconstructed skins for 10 days.
At the end of the treatment, the reconstructed skins were grown for a further period of 48 hours in the immersion medium and then the efficiency of the anti-oxidant treatment was assessed by: (1) analysis of cell viability (methylthiazoletetrazolium—MTT test), (2) flux cytometry dosage (CBA—Cytometric Bead Array) of the secretion of cytokines in the culture supernatants. This analysis is shown in the table below:

| | | | Reconstructed skin | Reconstructed skin |
|---|---|---|---|---|
| | Reconstructed skin | Reconstructed skin | UV irradiated + | UV irradiated + |
| | Control | UV irradiated | Flavagrum | Flavenge |
| MTT (viability) | 100% | 76% | 88% | 92% |
| IL1β (pg/mL) | 76 | 179 | 125 | 97 |
| IL6 (pg/mL) | 379 | 649 | 452 | 426 |
| IL8 (pg/mL) | 275 | 395 | 312 | 294 |
| TNFβ (pg/mL) | 53 | 152 | 105 | 89 |

With the inventive methods, it is possible to see that solar UV stress induces a reduction in the cell viability as well as an increase in the synthesis of pro-inflammatory interleukins. It is therefore of interest to limit this synthesis of pro-inflammatory molecules as well as cell mortality by using properly selected active ingredients. Among the screened active ingredients, two of them, Flavagrum and Flavenger, have demonstrated efficiency with a possible tendency to restore the reference level for both of these parameters.

### Example 25

### Use of the Three-Dimensional Pluricellular Reconstructed Skin Model Described in Examples 14, 17 and 20 for Investigating the Immunostimulating or Immunosupressive Activity of an Active Ingredient

In order to assess whether LCs and/or DCCs are capable of inducing immune and/or tolerogenic responses towards an active ingredient or not, the functionality of the cells to stimulate proliferation of allogenic naive T lymphocytes was investigated according to the following protocol:
Models were made according to the protocol described in Examples 14, 17 and 20.
On the 42^{nd} day of culture, either the active ingredient was directly applied on the reconstructed skins by means of a swab (topical application) or the active ingredient was introduced into the culture medium (systemic application) for 10 days.
At the end of the treatment, the cultures were successively digested by 2 hr enzymatic treatments at 37° C. with trypsin (1 mg/mL) and with collagenase (1 mg/mL) in order to recover the LCs and/or DCCs.
The LCs and/or DCCs were grown for 3 days with allogenic naive T lymphocytes in an RPMI culture medium supplemented with 10% human AB serum. A range of LCs and/or DCs between 125 and 8,000 cells was achieved and grown with 10⁵ naive T lymphocytes. On the 3^{rd} day of the mixed lymphocyte culture, 20 μl of 5 mCi tritiated thymidine was added for a period of 18 hrs.
The results are expressed in the table below with the number of LCs and/or DCCs in abscissae and in ordinates the incorporation of tritiated thymidine into allogenic naive T lymphocytes expressed in cpm (counts per minute):

| Number of LCs or DCCs | CPM (untreated) | CPM (active) |
|---|---|---|
| 125 | 500 | 500 |
| 250 | 800 | 1500 |
| 500 | 1000 | 10000 |
| 1000 | 1500 | 22500 |
| 2000 | 2300 | 39850 |
| 4000 | 2800 | 47250 |
| 6000 | 3000 | 52500 |
| 8000 | 3800 | 55000 |

After treatment with our active X, the LCs and/or DCCs strongly stimulated proliferation of T lymphocytes (between 5.10⁴ and 7.10⁴ cpm) as compared with the untreated LCs and/or DCCs which only induce weak proliferation of naive T lymphocytes (between 1.10³ and 4.10³ cpm).

### Example 26

### Use of Three-Dimensional Pluricellular Models for Performing Screening of Active Ingredients Capable of Modulating Allergic Reactions

The immunomodulating effect of an active ingredient after inducing an irritating, sensitizing and allergizing stress was investigated according to the following protocol:
The model was made according to the protocol described in Example 12.
After 21 days of culture, the irritant (sodium dodecyl sulphate) at the final concentration of 2% and the sensitizer DNCB (1,4-chloro-dinitrobenzene) at a final concentration of 0.25% were introduced into the culture medium for 24 hrs.
Next, 100 μl of a cosmetic formulation either containing a soothing active at the final concentration of 3% or not, was introduced into the culture medium of equivalent dermises for 3 days.
At the end of the treatment, the culture supernatants were recovered for dosing IL-10 and IL-12 for example, and an immunohistochemical study was developed with an antibody battery: anti-CD80, anti-CD83, anti-CD86, anti-CCR7, anti-DC-LAMP, anti-HLA-DR.

With the results of the IL-10 and IL-12 secretion and of the phenotype study of the DCCs by immunohistochemistry, the immunomodulating profile of the tested active ingredients was established. Thus, our pluricellular model is therefore a predictive tool with which it is possible to assess the potential immmunomodulating effect of an active ingredient by screening DCCs which have cytokine secretion, activation and maturation capabilities.

While the invention has been described in terms of various preferred embodiments, those skilled in the art will recognize that various modifications, substitutions, omissions and other changes may be made without departing from the spirit of the present invention.

1. A method for preparing Langerhans cells or interstitial dendritic cells or both Langerhans cells and interstitial dendritic cells from CD14⁺ monocytes, the method comprising
obtaining CD14⁺ monocytes from peripheral circulatory blood of a mammal; and
placing the monocytes into a cell culture environment comprising epithelial cells or mesenchymatous cells or both epithelial cells and mesenchymatous cells, but wherein exogenous cytokine is not added to the cell culture environment.

2. A method for growing CD14⁺ monocytes which differentiate into Langerhans cells, interstitial dendritic cells or both Langerhans cells and interstitial dendritic cells, the method comprising the integration in a cell or tissue model of CD14⁺ monocytes obtained from peripheral circulatory blood of a living mammal, wherein the cell or tissue model comprises epithelial cells or mesenchymatous cells or both epithelial cells and mesenchymatous cells.

3. The method of claim 2, wherein no exogenous cytokine is added.

4. The method of claim 2, wherein the cell or tissue model is selected from the group consisting of an epidermis model, an epithelium model, a dermis model, a chorion model, a skin model, and a mucosa model.

5. The method of claim 2, wherein the cell or tissue culture model comprises a dermal or chorion matrix support selected from the group consisting of:
a collagen- or fibrin-based gel or film comprising mesenchymatous cells;
a collagen-based porous matrix comprising chitosan or at least one glycosaminoglycan or both chitosan and at least one glycoaminoglycan;
an untreated inert support selected from the group consisting of a semi-permeable synthetic membrane; a semi-permeable nitrocellulose membrane; a semi-permeable nylon membrane; a polytetrafluoroethylene membrane or sponge; a semi-permeable polycarbonate or polyethylene terephthalate membrane; an inorganic membrane with a capillary porous structure of aluminum oxide; cellulose acetate or ester; a semi-permeable hydrophilized polytetrafluoroethylene; a semi-permeable polyester membrane; and an inert support treated for polycarbonate- or polystyrene-based culture.

6. The method of claim 5, wherein the matrix support is either a collagen-based porous matrix, a untreated inert support, or a treated support, and wherein mesenchymatous cells are integrated into the matrix support.

7. The method of claim 6, wherein the mesenchymatous cells are fibroblasts.

8. The method of claim 5, wherein epithelial cells are placed at the surface of the dermal or chorion matrix support.

9. The method of claim 8, wherein the epithelial cells are keratinocytes.

10. The method of claim 2, wherein the cell or tissue model comprises at least one additional cell type selected from the group consisting of nerve cells, Merkel cells, endothelial cells, macrophages, melanocytes, lymphocytes, adipose cells, or cutaneous annexes.

11. The method of claim 6, wherein a portion of the monocytes differentiate into endothelial cells or macrophages or both endothelial cells and macrophages.

12. The method of claim 5, wherein the cell or tissue model comprises an epithelial portion and a conjunctive matrix, and wherein a majority of Langerhans cells are localized in the epithelial portion and a majority of interstitial cells, macrophages and endothelial cells are localized in the conjunctive matrix.

13. A method for differentiating CD14⁺ monocytes into Langerhans cells or interstitial dendritic cells or both Langerhans cells and interstitial dendritic cells, the method comprising the following steps:
obtaining CD14⁺ monocytes from the circulatory blood of a mammal;
maintaining the monocytes under conditions that do not promote their differentiation into dendritic cells;
placing the monocytes into contact in a cell culture environment comprising epithelial cells or mesenchymatous cells or both epithelial cells and mesenchymatous cells under culture conditions promoting differentiation of the CD14⁺ monocytes into Langerhans cells or interstitial dendritic cells or both.

14. A cell model comprising a population of Langerhans cells or interstitial dendritic cells or both Langerhans cells and interstitial dendritic cells, wherein the Langerhans cell and interstitial dendritic cell populations are obtained by the method of claim 1.

15. The cell model of claim 14 further comprising a population of macrophages or endothelial cells.

16. A tissue model comprising a population of Langerhans cells or interstitial dendritic cells or both Langerhans cells and interstitial dendritic cells, wherein the Langerhans cell and interstitial dendritic cell populations are obtained by the method of claim 1, and wherein the tissue model is selected from the group consisting of an epidermis model, an epithelium model, a dermis model, a chorion model, a skin model, and a mucosa model.

17. The tissue model of claim 16 further comprising a population of macrophages or of endothelial cells.

18. The tissue model of claim 17 comprising an epithelial portion comprising epithelial cells and a conjunctive matrix comprising mesenchymatous cells, wherein the majority of Langerhans cells are localized in the epithelial portion and the majority of interstitial dendritic cells are localized in the conjunctive matrix.
